(19) United States
(12) Patent Application Publication
(10) Pub. No.: US 2014/0094657 A1
(43) Pub. Date: Apr. 3, 2014

(54) CONDITION CHECKING DEVICE FOR ENDOSCOPE

(71) Applicant: FUJIFILM Corporation, Tokyo (JP)

(72) Inventors: Manabu MIYAMOTO, Ashigarakami-gun (JP); Shinichi YAMAKAWA, Ashigarakami-gun (JP)

(73) Assignee: FUJIFILM Corporation, Tokyo (JP)

(21) Appl. No.: 14/038,359

(22) Filed: Sep. 26, 2013

(30) Foreign Application Priority Data
Sep. 28, 2012 (JP) 2012-217292

(51) Int. Cl. A61B 1/00; A61B 1/005

(52) U.S. Cl. CPC A61B 1/0008; A61B 1/0057
USPC 600/114; 600/117

(57) ABSTRACT

An endoscope system includes an endoscope, having a tip device for entry in a body cavity, and a viewing window portion formed in the tip device. A sleeve-shaped condition checking device is disposed on a distal side in an axial direction, for resiliently deforming in a transverse direction crosswise to the axial direction when pushed on an inner wall of the body cavity, to enter a viewing area of the viewing window portion. Furthermore, a propulsion assembly constitutes the condition checking device, and exerts force of propulsion to the tip device, for assistance to entry in the body cavity. The condition checking device includes a resilient end ring, disposed distally of a support sleeve, covered by a propulsion assembly, and having a tapered wall of which a diameter decreases in the axial direction from the support sleeve.

### BACKGROUND OF THE INVENTION

1. Field of the Invention

The present invention relates to a condition checking device for an endoscope. More particularly, the present invention relates to a condition checking device for an endoscope, which is utilized in the course of entry of the endoscope in a body cavity, and in which if push to an inner wall of a body cavity is carried out, the condition of the push can be visibly found.

2. Description Related to the Prior Art

An endoscope for imaging an inner wall of a body cavity is widely used for medical purpose and also for industrial use. The endoscope includes a handle and an elongated tube extending from the handle in a distal direction for entry in the body cavity. A tip device of the elongated tube has an imaging unit such as a CCD. A monitor display panel is driven to display an image according to an image signal generated by the imaging unit.

A propulsion assembly for assisting entry of the endoscope is known as an assist device mounted on the tip device of the endoscope. U.S. Pat. No. 2005/272,976 (corresponding to JP-A 2005-253892) discloses an example of the propulsion assembly including a support sleeve and an endless track device. The support sleeve is fastened to the tip device of the elongated tube of the endoscope. The endless track device is supported on the support sleeve in an endlessly movable manner. An outer surface of the endless track device is caused to contact the inner wall of the body cavity such as a gastrointestinal tract, to exert force to the tip device of the endoscope. This is effective in facilitating entry of the endoscope even into the body cavity with a highly tortuous form, such as a large intestine.

U.S. Pat. No. 8,177,709 (corresponding to JP-A2008-093029) discloses an endoscope system including the endoscope, the propulsion assembly and a drive mechanism. The propulsion assembly has a rotary tubular member, mounted on the elongated tube of the endoscope in a rotatable manner, and having a helical portion. The drive mechanism exerts rotational force to the rotary tubular member around an axial direction, and rotates the rotary tubular member to propel the elongated tube of the endoscope. The endoscope system includes a torque detector and a controller. The torque detector detects torque of the rotary tubular member. The controller receives an output from the torque detector, compares the detected torque with a torque limit predetermined for control of the rotary tubular member, and controls the drive mechanism according to a result of the comparison. If the detected torque of the rotary tubular member becomes higher than the torque limit, the controller controls the drive mechanism to stop the rotary tubular member or to decrease the torque of the rotary tubular member.

However, the propulsion assembly according to U.S. Pat. No. 2005/272,976 and U.S. Pat. No. 8,177,709 is disposed outside a viewing area of the endoscope for the purpose of reliable imaging without blocking. It is impossible for a doctor or operator visually to check failure in the advance of the endoscope due to push of the propulsion assembly to the inner wall of the body cavity for a long time. The propulsion assembly cannot be adjusted for smoothing the propulsion.

In the propulsion assembly of U.S. Pat. No. 8,177,709, the detected torque may become higher than the torque limit in the course of its increase due to the push of the rotary tubular member to the inner wall of the body cavity. However, friction may extremely increase according to the amount of the entry of the rotary tubular member, so that the detected torque may become higher than the torque limit in an apparently similar manner. It is impossible even according to the above-described control to find the condition of push of the propulsion assembly to the inner wall for a long time. It is necessary to stop or move backwards the endoscope, because the continued push of the propulsion assembly to the inner wall of the body cavity is unpreferable.

### SUMMARY OF THE INVENTION

In view of the foregoing problems, an object of the present invention is to provide a condition checking device for an endoscope, which is utilized in the course of entry of the endoscope in a body cavity, and in which if push to an inner wall of a body cavity is carried out, the condition of the push can be visibly found.

In order to achieve the above and other objects and advantages of this invention, a condition checking device for an endoscope having a tip device for entry in a body cavity, and a viewing window portion formed in the tip device, is provided. The condition checking device includes a sleeve-shaped view segment, disposed on a distal side in an axial direction, for resiliently deforming in a transverse direction crosswise to the axial direction when pushed on an inner wall of the body cavity, to enter a viewing area of the viewing window portion.

There is a propulsion assembly for constituting the view segment, and exerting force of propulsion to the tip device, for assistance to entry in the body cavity.

The propulsion assembly includes a coupling device for mounting on the tip device. A support sleeve is disposed around the coupling device. A resiliently deformable endless track device endlessly moves in the axial direction of the support sleeve by extending along inner and outer surfaces of the support sleeve.

The view segment includes a resilient end ring, disposed distally of the support sleeve, covered by the endless track device, and having a tapered wall of which a diameter decreases in the axial direction from the support sleeve.

The end ring is in a neck shape and includes a distal end wall, formed on a distal side of the tapered wall, and having a diameter increasing in the axial direction.

In another preferred embodiment, the view segment includes a resilient end ring, disposed distally of the coupling device, and having a tapered wall of which a diameter decreases in the axial direction from the coupling device.

The end ring is in a neck shape and includes a distal end wall, formed on a distal side of the tapered wall, and having a diameter increasing in the axial direction.

In one preferred embodiment, the view segment is constituted by the endless track device of a bag shape formed to extend in the axial direction longer than the support sleeve.

Furthermore, there is a motor. A rotatable wire component has a first end portion rotated by the motor, and a second end portion coupled to the propulsion assembly for driving the propulsion assembly.

In still another preferred embodiment, there is a hood component, mounted on the tip device, and having a tapered wall of which a diameter decreases in the axial direction from a proximal side.

Furthermore, a slit is formed in the hood component from a distal edge thereof, to extend in the axial direction.

Also, an endoscope system is provided, and includes an endoscope, having a tip device for entry in a body cavity, and a viewing window portion formed in the tip device. A sleeve-shaped condition checking device is disposed on a distal side in an axial direction, for resiliently deforming in a transverse direction crosswise to the axial direction when pushed on an inner wall of the body cavity, to enter a viewing area of the viewing window portion.

Furthermore, a propulsion assembly constitutes the condition checking device, and exerts force of propulsion to the tip device, for assistance to entry in the body cavity.

Consequently, if push is applied to an inner wall of a body cavity is carried out, the condition of the push can be visibly found, because a view segment of a condition checking device can be viewed through the viewing window portion.

### BRIEF DESCRIPTION OF THE DRAWINGS

The above objects and advantages of the present invention will become more apparent from the following detailed description when read in connection with the accompanying drawings, in which:

FIG. 1 is an explanatory view in a perspective, illustrating an endoscope and a condition checking device mounted on the endoscope;

FIG. 2 is a perspective view illustrating a tip device and the condition checking device;

FIG. 3 is an explanatory view in a block diagram, illustrating circuit elements of a controller;

FIG. 4 is a perspective view illustrating a propulsion assembly;

FIG. 5 is a perspective view illustrating the propulsion assembly;

FIG. 6 is a perspective view illustrating a mechanism for driving the propulsion assembly;

FIG. 7 is a vertical section illustrating the propulsion assembly;

FIG. 8 is a vertical section illustrating the tip device and the propulsion assembly mounted thereon;

FIG. 9A is a front elevation illustrating a viewing area of a viewing window portion;

FIG. 9B is a front elevation illustrating a condition with push of the condition checking device to an inner wall of the body cavity;

FIG. 10A is an explanatory view illustrating entry of the tip device in a rectum;

FIG. 10B is an explanatory view illustrating entry of the tip device in a sigmoid colon;

FIGS. 11A and 11B are explanatory views illustrating movement of the tip device in the sigmoid colon;

FIGS. 12A and 12B are explanatory views illustrating movement of the tip device in the sigmoid colon with a loop;

FIGS. 13A and 13B are explanatory views illustrating removal of the loop of the sigmoid colon by movement of the tip device;

FIGS. 14A and 14B are explanatory views illustrating movement of the tip device in a descending colon;

FIGS. 15A and 15B are explanatory views illustrating movement of the tip device in a transverse colon;

FIGS. 16A and 16B are explanatory views illustrating movement of the tip device in an ascending colon;

FIG. 17 is a vertical section illustrating another preferred condition checking device protruding from the support sleeve;

FIG. 18 is a vertical section illustrating one preferred condition checking device constituted by an endless track device;

FIG. 19 is a vertical section illustrating the condition checking device with push to the inner wall;

FIG. 20 is a perspective view illustrating still another preferred condition checking device constituted by a hood component;

FIG. 21A is a vertical section illustrating a condition without push of the condition checking device to an inner wall of the body cavity;

FIG. 21B is a vertical section illustrating a condition with push of the condition checking device to the inner wall;

FIGS. 22A, 22B, 22C and 22D are explanatory views illustrating the use of the condition checking device in the endoscope submucosal dissection (ESD).

### DETAILED DESCRIPTION OF THE PREFERRED EMBODIMENT(S) OF THE PRESENT INVENTION

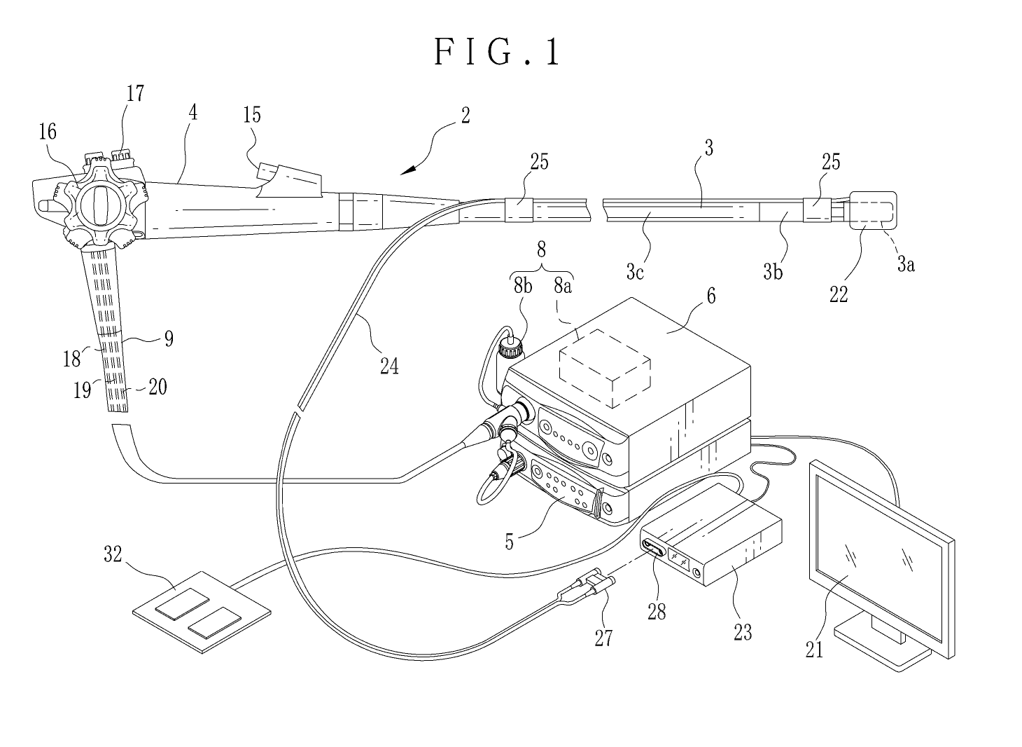

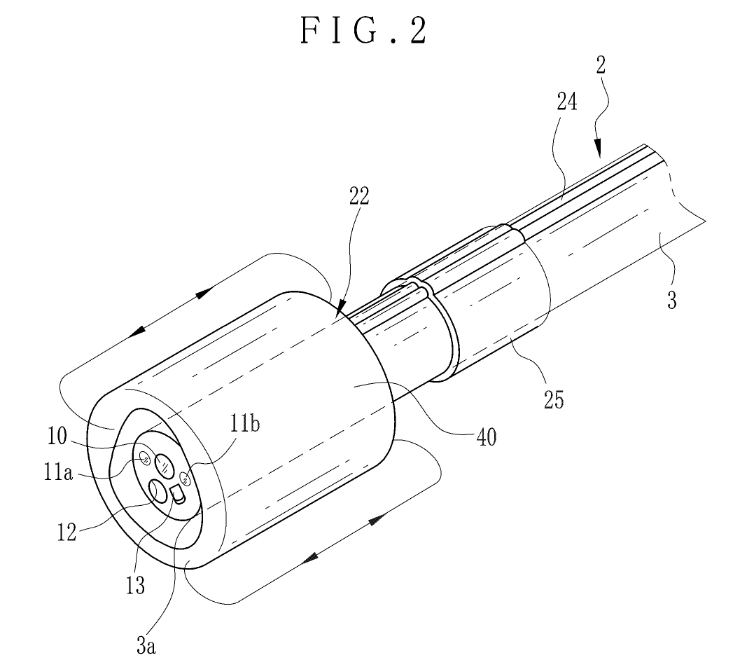

In FIGS. 1 and 2, an endoscope 2 for a medical use includes an elongated tube 3, a handle 4 and a universal cable 9. The elongated tube 3 is entered in a body cavity of a patient, such as a large intestine of a gastrointestinal tract. The handle 4 is used for holding the endoscope 2 and manipulating the elongated tube 3. The universal cable 9 connects the endoscope 2 to a processing apparatus 5, a light source apparatus 6 and a fluid supply source 8. The fluid supply source 8 is constituted by a pump 8a for supplying air, and a water reservoir 8b or tank. The pump 8a is a well-known device incorporated in the light source apparatus 6. The water reservoir 8b is disposed outside the light source apparatus 6, and stores water for washing.

The elongated tube 3 includes a tip device 3a, a steering device 3b and a flexible device 3c. The tip device 3a is rigid and includes an imaging unit to be described later. The steering device 3b extends to a proximal end of the tip device 3a and steerable up and down and to the right and left. The flexible device 3c is disposed between the steering device 3b and the handle 4.

The tip device 3a of the elongated tube 3 includes a viewing window portion 10, lighting window areas 11a and 11b and a distal instrument opening 12. A fluid nozzle 13 with a nozzle spout is formed in the tip device 3a for ejecting fluid to the viewing window portion 10, such as air and washing water. The lighting window areas 11a and 11b are so disposed that the viewing window portion 10 is positioned between those. The lighting window areas 11a and 11b emit light from the lighting apparatus toward an object of interest in the gastrointestinal tract.

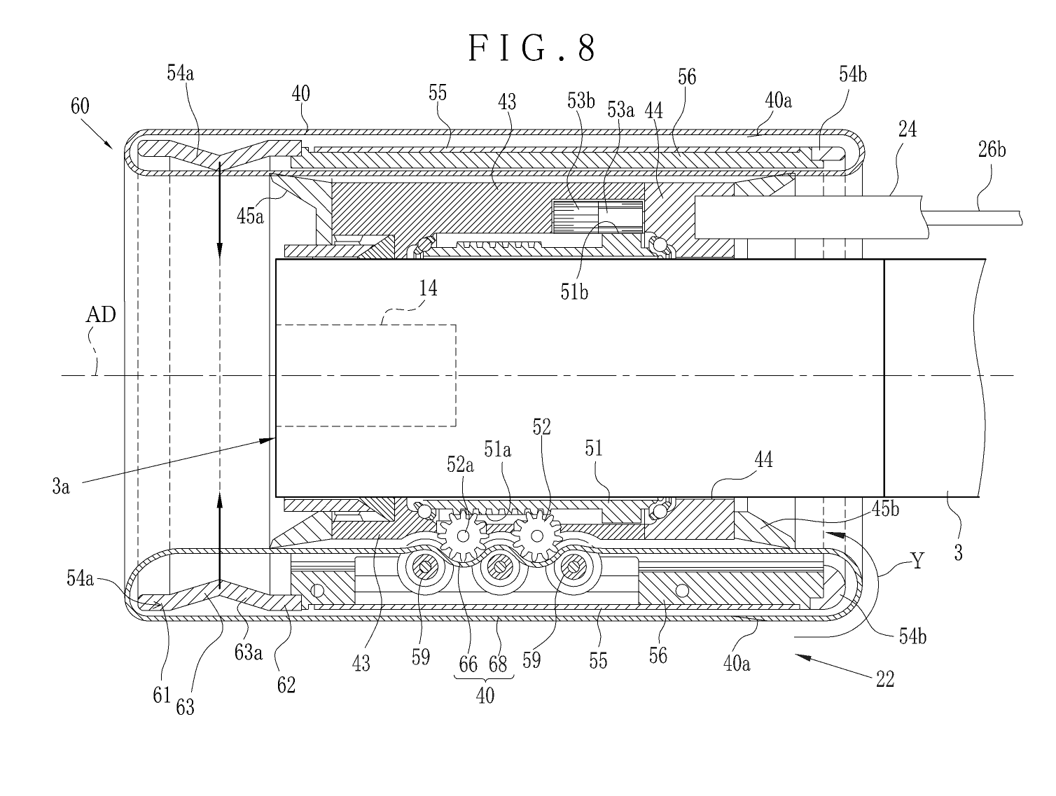

In FIG. 8, an imaging unit 14 is incorporated in the tip device 3a. The imaging unit 14 includes a lens system with the viewing window portion 10, and an image sensor, which is disposed behind the lens system and may be a CMOS or CCD image sensor as solid state imaging unit. Reflected light from the object of interest becomes incident upon the image sensor after passing the lens system with the viewing window portion 10. A proximal instrument opening 15 is formed in the handle 4. An instrument channel extends from the distal instrument opening 12 to the proximal instrument opening 15. Various medical instruments are entered in the proximal instrument opening 15 for treatment or diagnosis, for example, a forceps, injection needle, high frequency surgical instrument, and the like.

The handle 4 includes steering wheels 16 and a fluid button 17. The steering wheels 16 are rotatable for steering the steering device 3b up and down and to the right and left. The fluid button 17 is depressed for supplying air or water or sucking body fluid. The universal cable 9 is connected to the handle 4. The universal cable 9 contains a fluid tube 18, a signal line 19 and a light guide device 20. A proximal end of the fluid tube 18 is connected to the fluid supply source 8. A distal end of the fluid tube 18 is connected to the fluid nozzle 13, so that the fluid tube 18 supplies air or water from the fluid supply source 8 to the fluid nozzle 13.

A proximal end of the signal line 19 is connected to the processing apparatus 5. A distal end of the signal line 19 is connected to a CCD image sensor, for transmitting a control signal and an image signal. A distal end of the light guide device 20 is connected to the lighting window areas 11a and 11b. A proximal end of the light guide device 20 is connected to the light source apparatus 6 and transmits light from the light source apparatus 6 to the lighting window areas 11a and 11b. The processing apparatus 5 processes the image signal input from the signal line 19 in signal processing of suitable functions. A monitor display panel 21 is driven to display an image according to the image signal.

A propulsion assembly 22 is mounted on the tip device 3a of the elongated tube 3 for moving the elongated tube 3 back and forth in the gastrointestinal tract. An actuating unit 23 actuates the propulsion assembly 22.

The actuating unit 23 is electrically connected to the processing apparatus 5. A protection sheath 24 of a flexible form extends from a proximal end of the propulsion assembly 22, and includes two parallel sheath portions. An adhesive tape 25 or surgical tape attaches the protection sheath 24 to plural points on the elongated tube of the endoscope. The protection sheath 24 is prevented from moving irregularly in a body cavity upon entry or manipulation of the endoscope with the propulsion assembly 22.

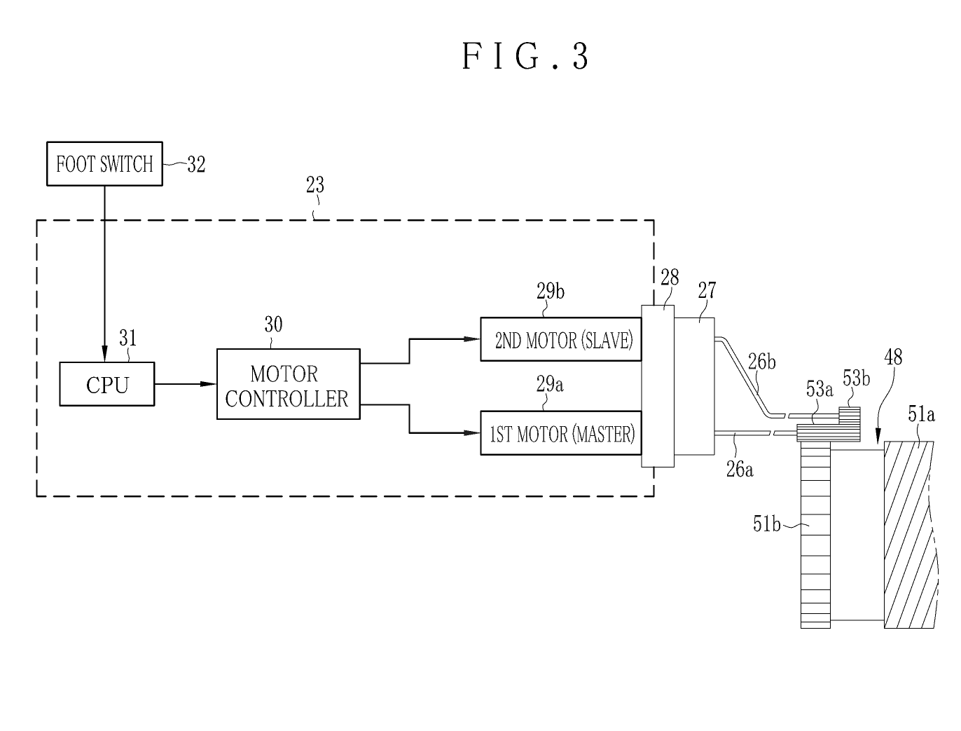

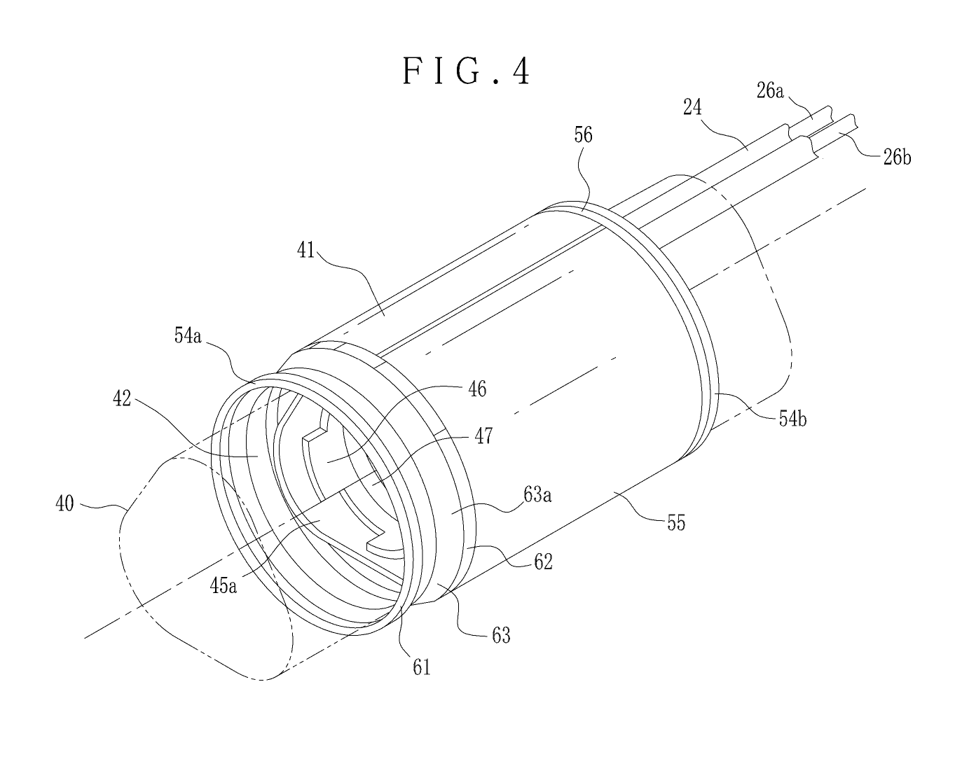

A first wire component 26a or master wire component, and a second wire component 26b or slave wire component (See FIG. 4) are entered through the protection sheath 24, and have distal tips which are mechanically coupled to the propulsion assembly 22. The first and second wire components 26a and 26b have high flexibility and also high rigidity to torsion. Torque input for proximal tips of the first and second wire components 26a and 26b is transmitted by those without attenuation. A receptacle connector 28 is provided in the actuating unit 23. A connection plug 27 of a fork shape couples proximal tips of the first and second wire components 26a and 26b to the receptacle connector 28. A first motor 29a or master motor and a second motor 29b or slave motor (See FIG. 3) are incorporated in the actuating unit 23. When the connection plug 27 is coupled to the receptacle connector 28, the first wire component 26a becomes rotatable by the first motor 29a, and the second wire component 26b becomes rotatable by the second motor 29b.

In FIG. 3, the actuating unit 23 includes a motor controller 30 and a CPU 31. A rotational speed of the first motor 29a is set at 2,000 rpm by the control with a current from the motor controller 30. There is a foot switch 32 with which the motor controller 30 changes over the turn-on and turn-off states and forward and backward rotations of the first and second motors 29a and 29b.

The propulsion assembly 22 is utilized typically with the endoscope 2 for the large intestine for the purpose of assisting the advance and return typically in the sigmoid colon and the transverse colon. The propulsion assembly 22 includes an endless track device 40 (membrane) for contacting an inner wall of the gastrointestinal tract for exerting force for the advance and return to the elongated tube 3 of the endoscope 2. The endless track device 40 has a shape with a cylindrical profile and with an outer surface of a toroid form, and is formed from a resiliently deformable sheet material. The endless track device 40 is movable endlessly in the axial direction AD.

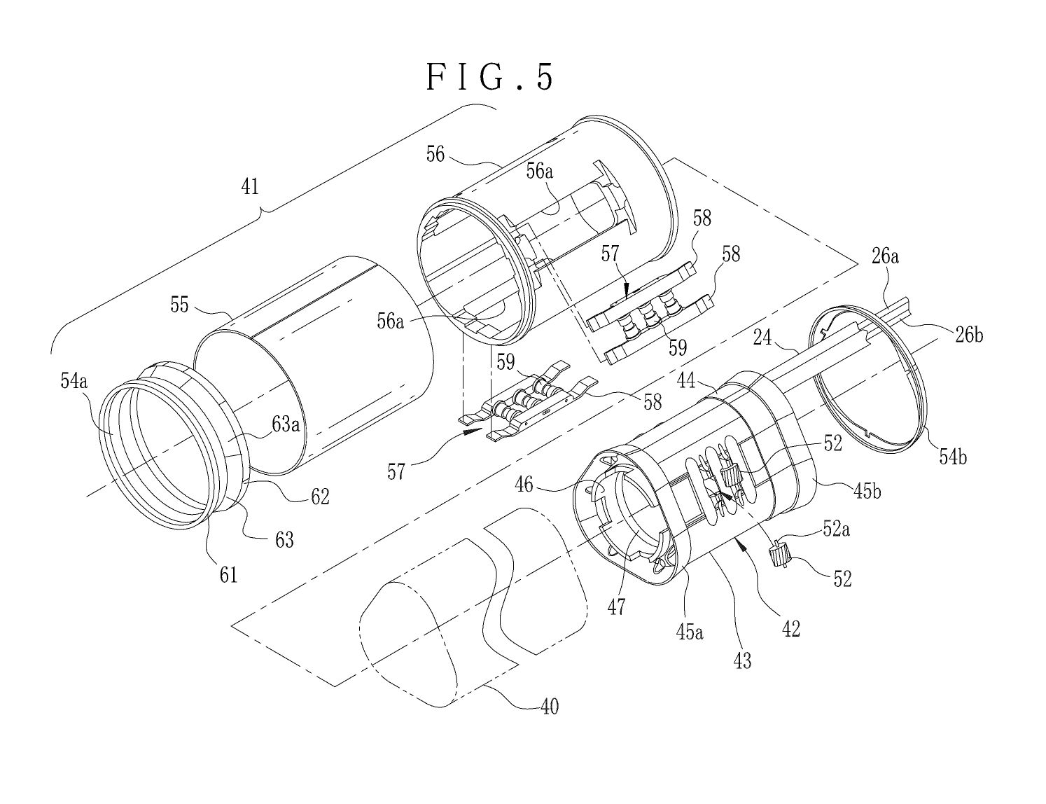

In FIGS. 4-6, there is a barrel unit 41 or outer sleeve unit having inner and outer surfaces, along which the endless track device 40 extends and moves endlessly in an axial direction. In FIGS. 4 and 5, a developed form of the endless track device 40 is illustrated for structural simplicity. For a final form of the endless track device 40, proximal and distal ends of a tubular material of the developed form are bent back externally, and are attached to one another by thermal welding. Thus, the endless track device 40 becomes shaped in a bag form as if a doughnut form were extended along its hole. Note that the endless track device 40 can be formed by molding by use of a mold set. Note that in FIGS. 4-7, a left end of the tip device 3a is the distal end. A right end of the tip device 3a is the proximal end directed to the handle 4.

The endless track device 40 is formed from deformable material with flexibility, and compressibility and/or expandability. Examples of the material are polyvinyl chloride, polyamide resin, fluorocarbon resin, urethane, polyurethane, and other biocompatible plastic materials.

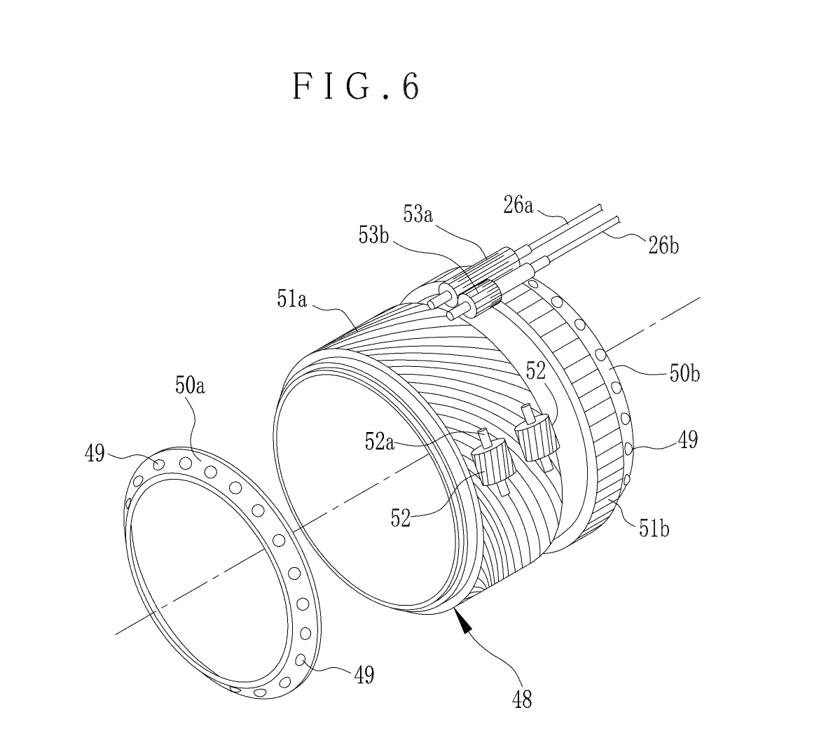

A drive unit 42 or inner sleeve unit is disposed in the endless track device 40 and the barrel unit 41. The drive unit 42 includes a carrier sleeve 43 (inner support sleeve), a cap ring 44, a distal cover flange 45a, a proximal cover flange 45b, a clamping device 46, a C-ring 47 or coupling device, and a drive sleeve 48. The carrier sleeve 43 has a cylindrical receiving hole, and an outer surface in a shape of a triangular prism. The cap ring 44 is triangular and attached to a proximal end of the carrier sleeve 43 with screws, press-fit, caulking or the like. The cover flanges 45a and 45b are fixed respectively to a distal end of the carrier sleeve 43 and a proximal end of the cap ring 44. The clamping device 46 is helically engaged with an inner thread inside the carrier sleeve 43, and moved axially upon being rotated. The C-ring 47 is formed from synthetic resin, and has a diameter increasing and decreasing upon movement of the clamping device 46 in the axial direction. The drive sleeve 48 is supported in the carrier sleeve 43 rotatably. See FIG. 6.

In FIG. 6, there are bearing rings 50a and 50b on which bearing balls 49 are supported in an annular form. The drive sleeve 48 is supported inside the carrier sleeve 43 with the bearing rings 50a and 50b in a rotatable manner, and is prevented from drop by the cap ring 44 fixedly engaged with a proximal end of the carrier sleeve 43. Teeth of a worm gear 51a and a spur gear 51b are formed on an outer surface of the drive sleeve 48. A pair of drive wheels 52 or worm wheels are supported on the carrier sleeve 43 in a rotatable manner, and are meshed with the worm gear 51a through an opening formed in the carrier sleeve 43. The drive wheels 52 are arranged in three positions, and rotate about their gear shafts 52a in an equal direction when the drive sleeve 48 rotates.

A distal end of the protection sheath 24 is attached to the inside of a recess formed on a proximal side of the cap ring 44 by use of adhesion or thermal welding. Ends of the first and second wire components 26a and 26b protrude from the distal end of the protection sheath 24, penetrate in through holes in the cap ring 44, and extend distally of the cap ring 44. A first pinion 53a and a second pinion 53b are fixedly secured to the first and second wire components 26a and 26b. As depicted in the drawing, shafts protrude from ends of the pinions 53a and 53b as rotational centers, and are entered through holes formed in the carrier sleeve 43, so that the pinions 53a and 53b are respectively supported in a rotatable manner. Among the pinions 53a and 53b, the first pinion 53a on the first wire component 26a is meshed with the spur gear 51b of the drive sleeve 48. The second pinion 53b on the second wire component 26b is meshed with the first pinion 53a but not with the spur gear 51b. The drive sleeve 48 is driven by rotations of the first pinion 53a with the first wire component 26a. Each of the first and second wire components 26a and 26b is driven by rotational force discretely supplied by the actuating unit 23. The second pinion 53b is rotated in a direction reverse to that of the first pinion 53a. Thus, rotational force of the second wire component 26b is added to the rotational force of the first pinion 53a, to rotate the drive sleeve 48 at a high torque.

Each of the cover flanges 45a and 45b has a flange edge directed with a larger diameter, for contacting an inner surface of the endless track device 40 moved endlessly. The cover flanges 45a and 45b prevent dust, tissue of the body cavity and the like from entry in the propulsion assembly 22 together with movement of the endless track device 40.

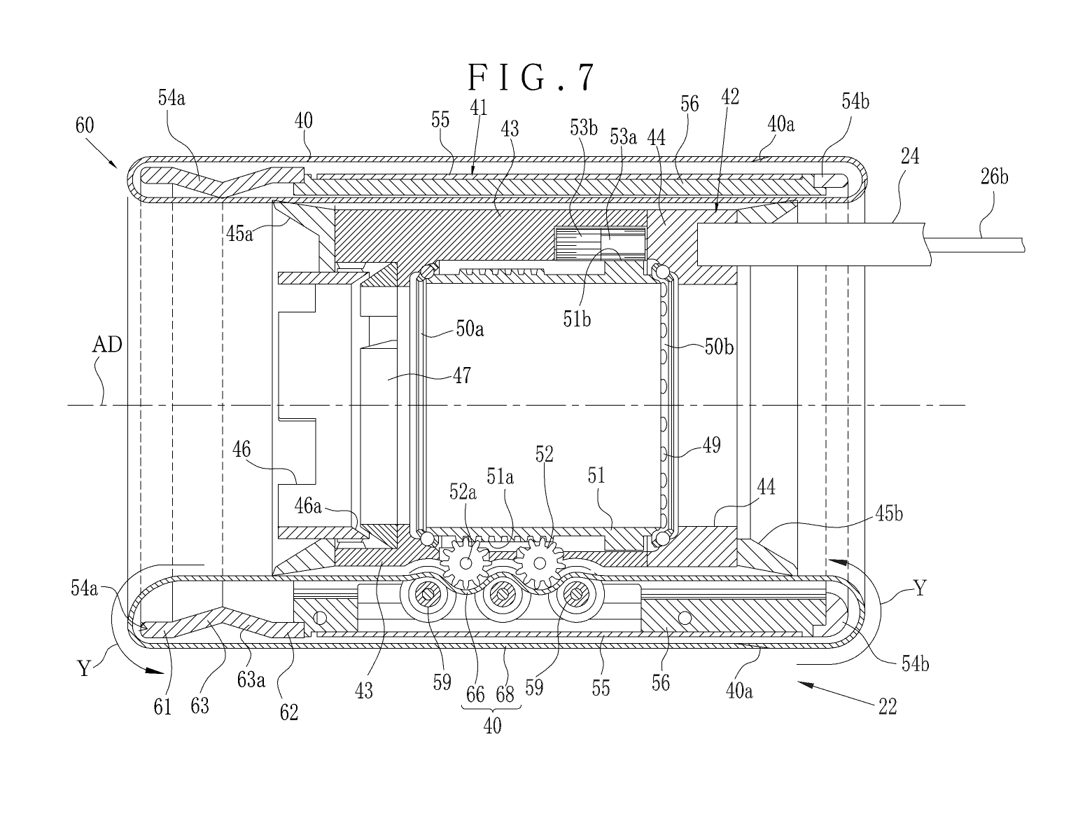

A distal end of the clamping device 46 has engagement teeth or the like arranged regularly in a circumferential direction. A tool is entered through the distal end and can be engaged with the engagement teeth of the clamping device 46. The clamping device 46, when rotated in a direction for helical engagement by the tool, is moved toward a proximal side axially. An inner tapered surface 46a of the clamping device 46 of FIG. 7 presses the C-ring 47 and deforms the same to decrease its diameter. The tip device 3a of the endoscope is entered in the receiving hole of the carrier sleeve 43 before the clamping device 46 is rotated for helical engagement. Then the inner surface of the C-ring 47 is pressed on the outer surface of the tip device 3a, to which the carrier sleeve 43 can be fastened reliably.

The barrel unit 41 includes a distal end ring 54a, a shield cover 55, a support sleeve 56 and a proximal end ring 54b. Elements of the barrel unit 41 are assembled to connect the drive unit 42 with the endless track device 40 according to the following steps.

In FIGS. 4 and 5, the drive unit 42 is positioned in the developed form of the endless track device 40 to cover the outer surface of the drive unit 42 with various elements. Then the drive unit 42 with the endless track device 40 is entered in a receiving hole of the support sleeve 56. Three quadrilateral openings 56a are formed in the support sleeve 56 and arranged at a pitch of 120 degrees circumferentially. Roller units 57 are fitted in respectively the quadrilateral openings 56a.

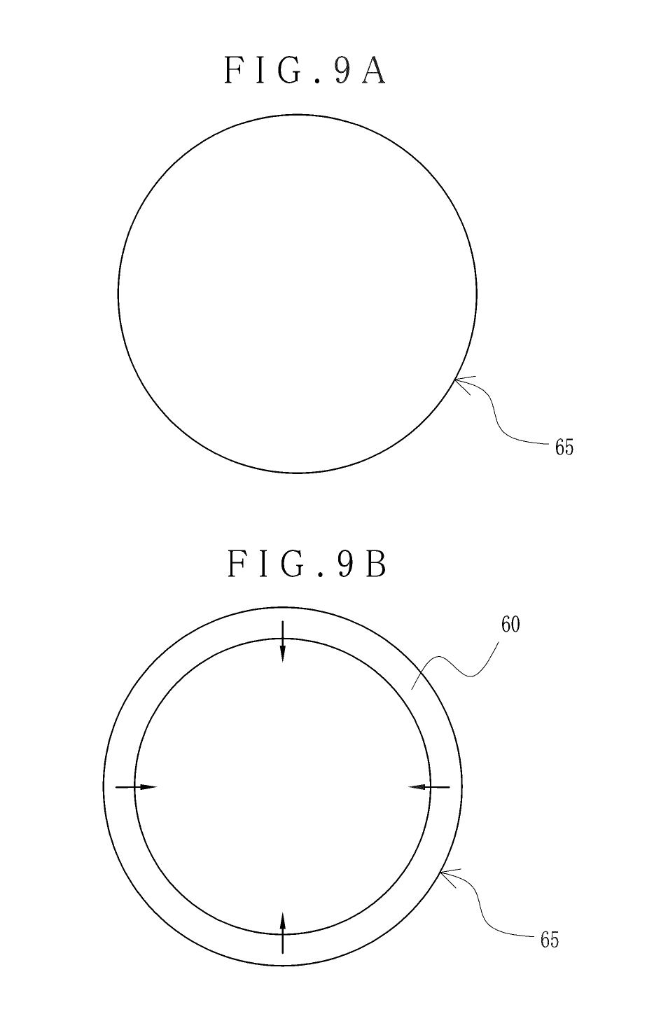

Each of the roller units 57 includes a pair of holder frames 58 and three idler rollers 59 supported between the holder frames 58. The holder frames 58 are formed from thin plates of metal with resiliency. End grooves for engagement are formed with ends of the quadrilateral openings 56a. Ends of the holder frames 58 are engaged with the end grooves. A center portion of the holder frames 58 in the longitudinal direction is curved to enter a center space in the support sleeve 56. The holder frames 58 are curved so that the idler rollers 59 on the holder frames 58 push the endless track device 40 to the drive wheels 52. In FIGS. 9A and 9B, the endless track device 40 is tightly tensioned between the drive wheels 52 and the idler rollers 59.

After the roller units 57 are fitted in the quadrilateral openings 56a, the support sleeve 56 is not movable in the axial direction relative to the drive unit 42, because the idler rollers 59 protrude internally from the inner surface of the support sleeve 56. The idler rollers 59 are combined to tension the endless track device 40. Also, the end rings 54a and 54b are attached to the support sleeve 56. The shield cover 55 is fitted on the outer surface of the support sleeve 56 for tightly covering the support sleeve 56 and the roller units 57.

A developed sheet of the endless track device 40 in a tubular shape is positioned between the drive unit 42 and the barrel unit 41, which are combined together. Front and rear ends of the developed sheet are bent back externally to join the rear end to the front end. The front and rear ends can have inclined surfaces, according to which connected portions 40a of the front and rear ends can be free from large irregularity in the thickness. FIG. 7 is a section schematically illustrating the propulsion assembly 22 after being assembled. The endless track device 40 comes to have an inner space for containing the barrel unit 41 entirely. It is possible to charge the inner space with air, physiological saline water, synthetic resin of a colloid condition, lubricant such as oil or grease, or other suitable substances.

The endless track device 40 is formed by attachment of the ends of the tubular sheet, and is in a bag form of FIG. 7. The endless track device 40 is tensioned between the drive wheels 52 and the idler rollers 59. Rotations of the drive wheels 52 are transmitted to the endless track device 40 which can be moved in the axial direction.

A condition checking device 60 (end flange for visual aid) with a view segment (distal extension) is constituted by the distal end ring 54a with the endless track device 40. As will be described later, the condition checking device 60 is deformed resiliently when the propulsion assembly 22 is pushed on the inner wall of the body cavity, and enters the viewing area of the imaging unit 14 in a deformed state.

The distal end ring 54a includes a distal end wall 61, a proximal end wall 62 and a neck portion 63. The proximal end wall 62 has inner and outer diameters equal to those of the distal end wall 61. The neck portion 63 is disposed between the end walls 61 and 62. The distal end ring 54a is a resilient device formed from silicon rubber, fluororubber, polyurethane and the like. The neck portion 63 includes a tapered wall 63a having a diameter decreasing in a distal direction from the proximal side. In the embodiment, the condition checking device 60 extends so that its axis is aligned with the axial direction of the tip device 3a upon mounting the propulsion assembly 22 thereon. The axis of the neck portion 63 is aligned with the optical axis of the imaging unit 14.

When the propulsion assembly 22 with the tip device 3a is entered in a body cavity and pushed on its inner wall, the neck portion 63 is resiliently deformed with the endless track device 40 (by way of the condition checking device 60 together with the distal end ring 54a) in a direction transverse to the axial direction to enter the viewing area of the imaging unit 14.

In FIG. 9A, the condition checking device 60 is not pushed on the inner wall of the body cavity. The condition checking device 60 is located outside a viewing area 65 of the imaging unit 14. When the condition checking device 60 is pushed on the inner wall, the neck portion 63 is deformed radially to decrease its inner diameter. As described heretofore, the neck portion 63 is disposed coaxially with the imaging unit 14. As illustrated in FIG. 9B, the condition checking device 60 enters the viewing area 65 at an equal width circumferentially when pushed on the inner wall.

The operation of the propulsion assembly 22 is described now. The propulsion assembly 22 is mounted on the tip device 3a by positioning the condition checking device 60 distally of the tip device 3a. A special device is used for mounting the propulsion assembly 22, and rotates the clamping device 46 in a clockwise direction. As the clamping device 46 is helically engaged with the inner thread formed on the inner surface of the carrier sleeve 43 on the distal side, the clamping device 46 rotates in a clockwise direction and moves in the proximal direction. The inner tapered surface 46a presses the C-ring 47. The tapered surface is formed on the distal side of the C-ring 47, and pushed by the inner tapered surface 46a of the clamping device 46 to deform the C-ring 47 resiliently to decrease its diameter. Upon the deformation, the tip device 3a is squeezed by the C-ring 47 to fasten the propulsion assembly 22 on the tip device 3a tightly.

The protection sheath 24 drawn from the proximal end of the propulsion assembly 22 is extended along the surface of the flexible device from the steering device. The plural indicia are present on the surface of the protection sheath 24 for indicating the positions for attachment of a tape at a suitable interval. The adhesive tape 25 is utilized to attach the protection sheath 24 on the steering device and flexible device of the endoscope at the indicia. The connection plug 27 at a proximal end of the protection sheath 24 is entered in the receptacle connector 28 and coupled to the actuating unit 23. A power source for the actuating unit 23 is turned on.

When the imaging is ready as described above, the tip device 3a of the endoscope 2 is entered in a body cavity, for example, large intestine. The foot switch 32 in connection with the actuating unit 23 is operated. The CPU 31 controls the motor controller 30 to supply the first and second motors 29a and 29b with a current according to a rotational speed by use of the motor controller 30. The first and second motors 29a and 29b are driven to rotate the first and second wire components 26a and 26b. In response, the pinions 53a and 53b are rotated. The drive sleeve 48 is rotated in cooperation with the spur gear 51b meshed with the first pinion 53a. The second pinion 53b is rotated in a direction reverse to that of the first pinion 53a. Rotations of the second pinion 53b are transmitted to the first pinion 53a. Thus, the first and second motors 29a and 29b are utilized together in the actuating unit 23 to rotate the drive sleeve 48.

When the worm gear 51a rotates together with the drive sleeve 48, the drive wheels 52 are rotated in an equal direction respectively about the gear shafts 52a. A return run 66 of the endless track device 40 is tensioned tightly between the tooth surface of the drive wheels 52 and the idler rollers 59 of the roller units 57. Thus, the idler rollers 59 are rotated by rotations of the drive wheels 52, to move the endless track device 40 in the axial direction of the drive sleeve 48.

When the tip device 3a of the endoscope 2 enters the large intestine with the propulsion assembly 22 and a working run 68 of the endless track device 40 contacts the inner wall, the propulsion force for moving the tip device 3a forwards is obtained during the endless movement of the endless track device 40. In other words, force exerted to the inner wall in the proximal direction is obtained.

Light from the light source apparatus 6 travels through the light guide device 20 and the lighting window areas 11a and 11b and is applied to the inside of the large intestine. The imaging unit 14 in the tip device 3a outputs an image signal by imaging the inner wall of the large intestine. The image signal is transmitted by the signal line 19 in the endoscope 2 and input to the processing apparatus 5, for the display panel 21 to display an image. A doctor or operator views the inner wall by use of the display panel 21.

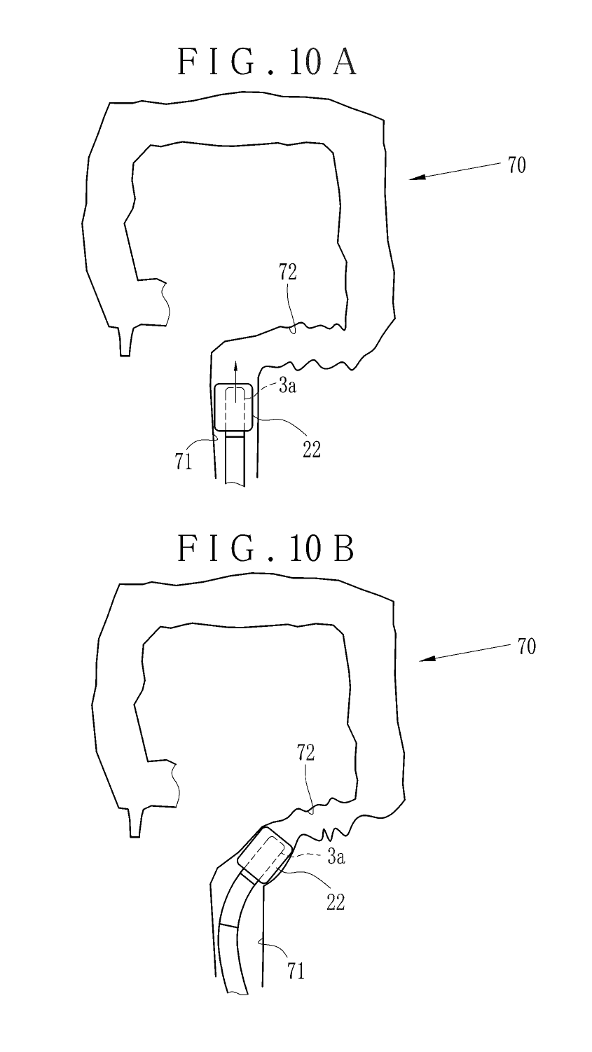

The operation of the propulsion assembly 22 for imaging a large intestine 70 is described now by referring to FIGS. 10A-16B. At first, the doctor or operator enters the tip device 3a with the propulsion assembly 22 into a rectum 71 through the anus as illustrated in FIG. 10A. After the entry, the foot switch 32 is manipulated to move the endless track device 40 endlessly in a direction to advance the propulsion assembly 22 and the tip device 3a. The propulsion assembly 22 and the tip device 3a reach a sigmoid colon 72 after the advance from the rectum 71 as illustrated in FIG. 10B.

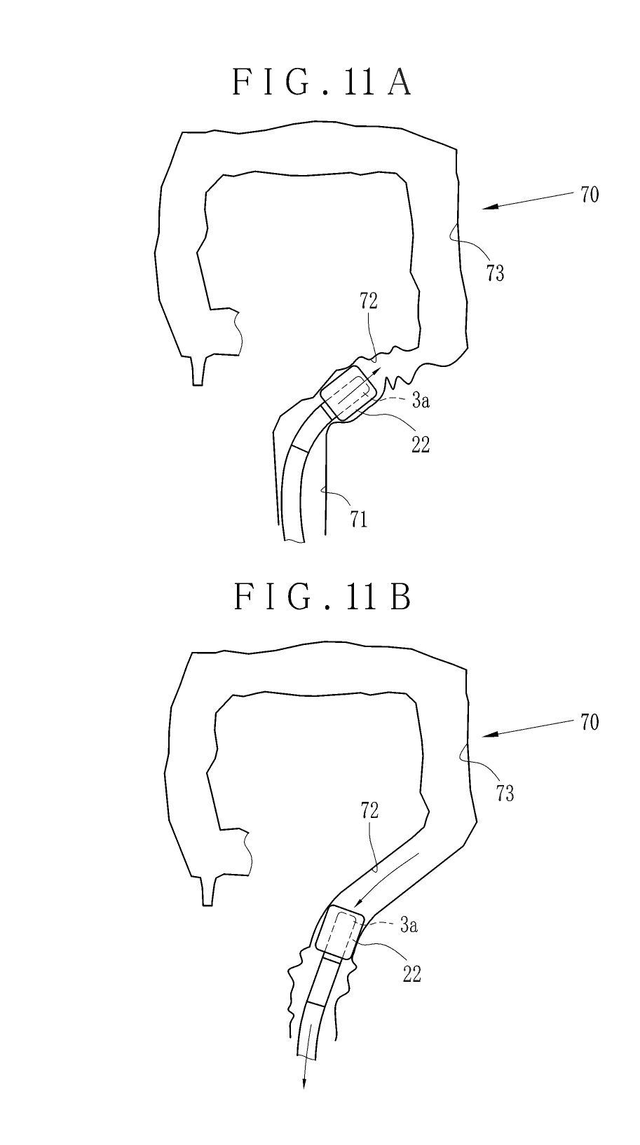

The sigmoid colon 72 is a mobile part of the gastrointestinal tract with looseness, namely, is not attached to the body. When the propulsion assembly 22 and the tip device 3a enter the sigmoid colon 72, the doctor or operator endlessly moves the endless track device 40 in a direction of advance as much as 10-20 cm. See FIG. 11A. Then the elongated tube 3 is returned by pull from the body cavity in FIG. 11B at an amount of the advance of the propulsion assembly 22 and the tip device 3a. Thus, the sigmoid colon 72 with the looseness can be drawn toward the rectum 71. Similarly, the step of advancing the propulsion assembly 22 and the tip device 3a and the step of pulling the elongated tube 3 are repeated alternately, to straighten the sigmoid colon 72. A lower end of a descending colon 73 becomes visible beyond the sigmoid colon 72 being straight. He or she sees the display panel 21, and advances the propulsion assembly 22 and the tip device 3a to pass the sigmoid colon 72 by viewing the lower end of the descending colon 73 in the viewing area.

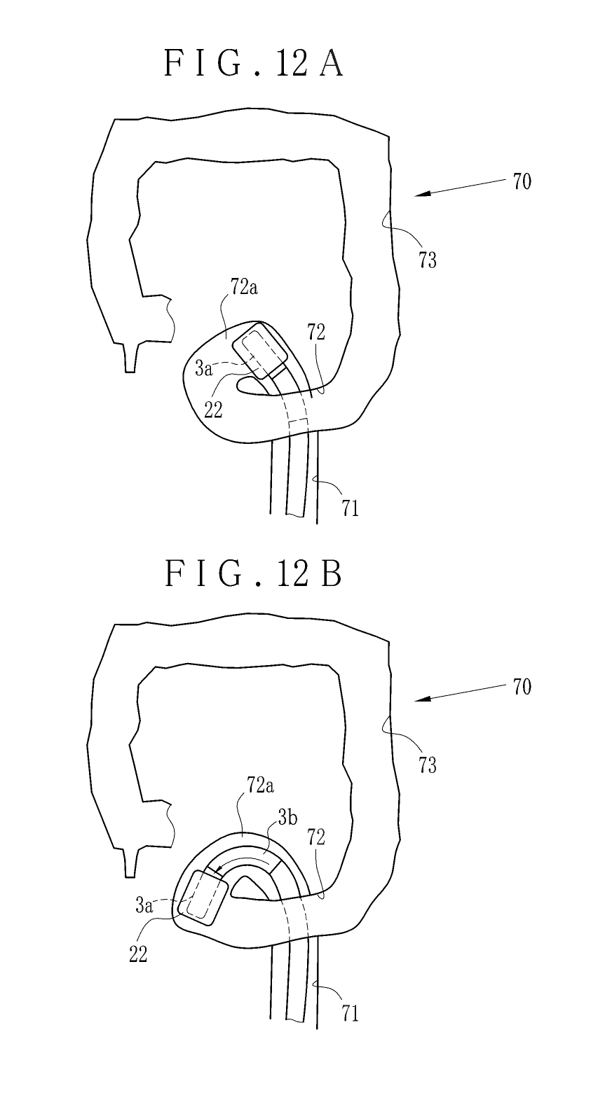

In FIGS. 12A and 12B, a loop 72a of the sigmoid colon 72 may occur typically when the sigmoid colon 72 has a great length and looseness. For entry into the sigmoid colon 72, at first the propulsion assembly 22 and the tip device 3a are moved forwards along the tortuous form of the sigmoid colon 72 as illustrated in FIG. 12A. The doctor or operator views the display panel 21, and rotates the steering wheels 16 to steer the steering device 3b in a direction of the tortuous form of the sigmoid colon 72. See FIG. 12B.

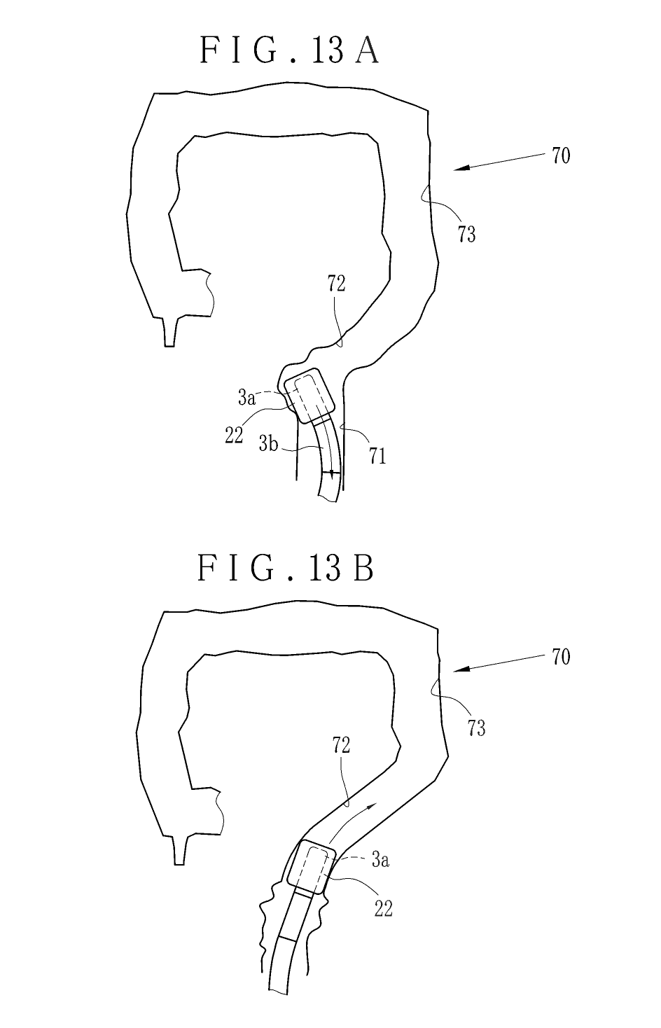

The steering device 3b is sufficiently steered according to the tortuous form of the sigmoid colon 72. He or she returns the elongated tube 3 as long as 20-25 cm. The steering device 3b is also returned to a straight form. See the state of FIG. 13A. The loop 72a of the sigmoid colon 72 is removed gradually for a straight form. He or she sees the display panel 21 and finds the straight form of the sigmoid colon 72. Then it is possible to advance the propulsion assembly 22 and the tip device 3a in the manner similar to the above. See the state of FIG. 13B.

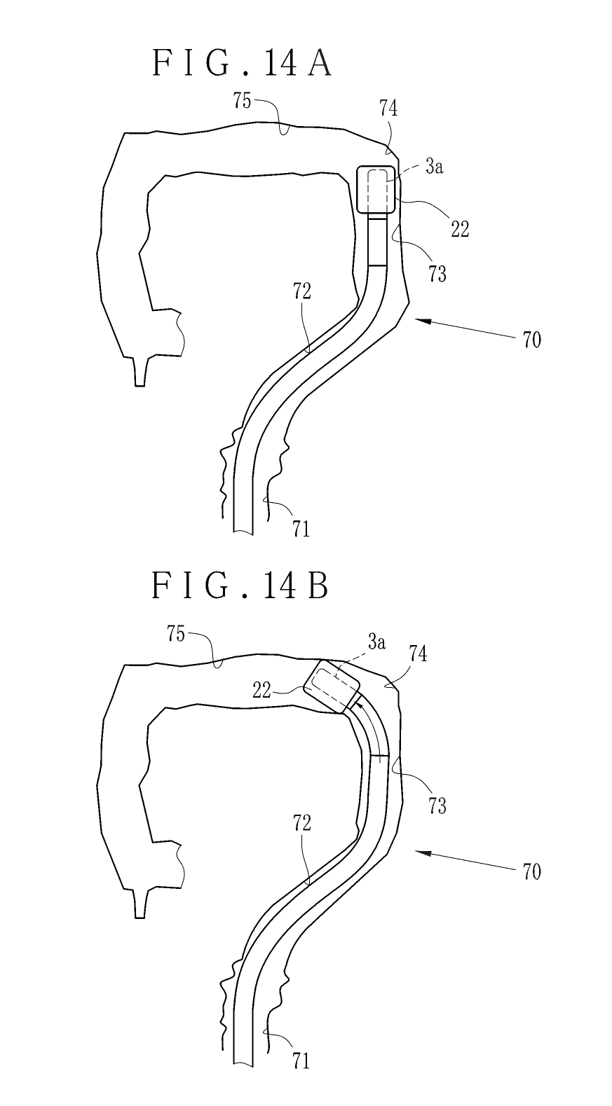

When the propulsion assembly 22 and the tip device 3a pass the sigmoid colon 72 and enter the descending colon 73, a splenic flexure 74 comes to appear ahead of the tip device 3a as illustrated in FIG. 14A. The doctor or operator views the splenic flexure 74 in the viewing area in the display panel 21, and moves the propulsion assembly 22 and the tip device 3a distally to pass the descending colon 73.

When the propulsion assembly 22 and the tip device 3a reach the splenic flexure 74 beyond the descending colon 73, the doctor or operator manipulates the steering wheels 16 by viewing the display panel 21. The steering device 3b is steered to seek for a direction of a transverse colon 75 beyond the splenic flexure 74. Then the propulsion assembly 22 and the tip device 3a are advanced. The steering device 3b is steered according to a direction of the bend of the splenic flexure 74. The propulsion assembly 22 and the tip device 3a are advanced and can pass the splenic flexure 74 reliably. See FIG. 14B.

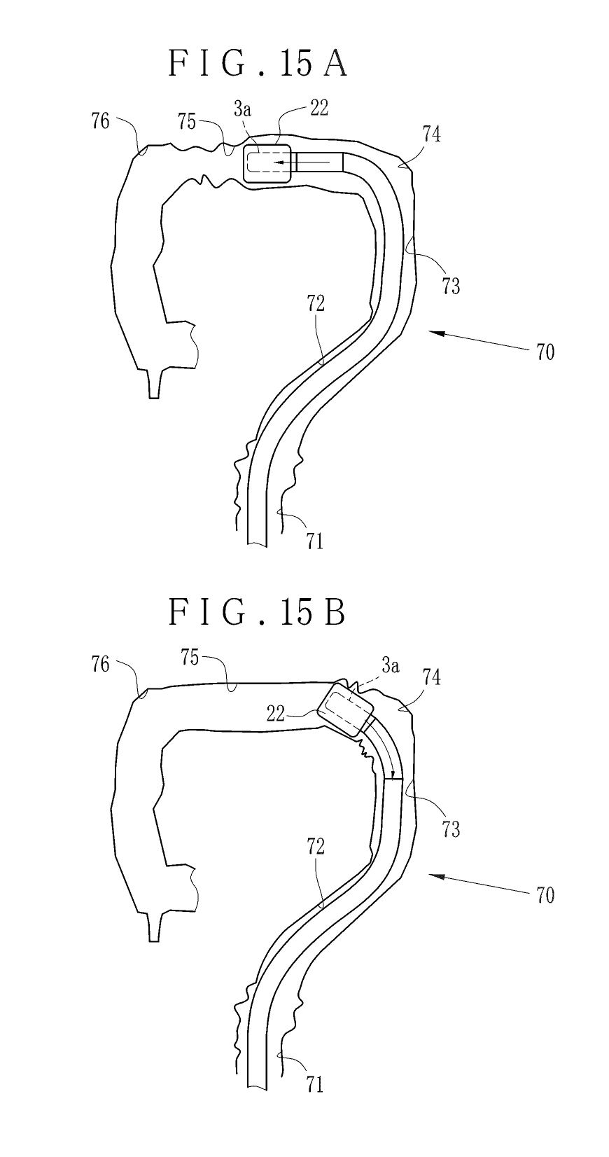

When the propulsion assembly 22 and the tip device 3a are moved to pass the splenic flexure 74 and enter the transverse colon 75, the operator rotates the steering wheels 16 to return the steering device 3b. The transverse colon 75 is not attached to the body, but is mobile in a manner similar to the sigmoid colon 72. Upon entry of the propulsion assembly 22 and the tip device 3a in the transverse colon 75, the operator repeats the advance of the propulsion assembly 22 and the tip device 3a (See FIG. 15A) and the return of the elongated tube 3 (See FIG. 15B), to extend the transverse colon 75 straight in a manner similar to the sigmoid colon 72. Then a hepatic flexure 76 appears ahead of the tip device 3a.

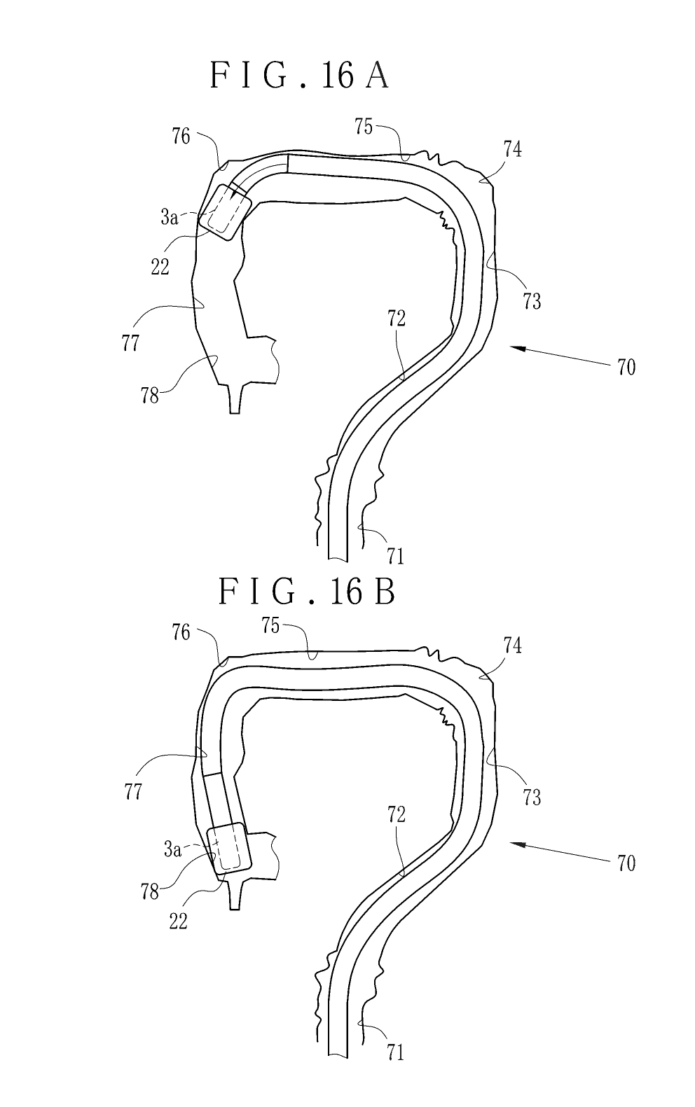

When the propulsion assembly 22 and the tip device 3a reach the hepatic flexure 76 beyond the transverse colon 75, the doctor or operator manipulates the steering wheels 16 by viewing the display panel 21 again. The steering device 3b is steered to seek for a direction of an ascending colon 77 beyond the hepatic flexure 76. Then the propulsion assembly 22 and the tip device 3a are advanced. The steering device 3b is steered according to a direction of the bend of the hepatic flexure 76. The propulsion assembly 22 and the tip device 3a are advanced and can pass the hepatic flexure 76 reliably. See FIG. 16A.

Upon the entry of the propulsion assembly 22 and the tip device 3a in the ascending colon 77 beyond the hepatic flexure 76, the steering wheels 16 are rotated to set the steering device 3b in a straight form. After the reach to the ascending colon 77, a cecum 78 becomes viewed. The propulsion assembly 22 and the tip device 3a are advanced to reach the cecum 78 as illustrated in FIG. 16B.

As described heretofore, the sigmoid colon 72 and the transverse colon 75 are mobile (not attached) in the body, and failure is likely to occur in the smooth advance of the propulsion assembly 22 for the purpose of imaging of the large intestine 70. It is likely that the propulsion assembly 22 is pushed on the inner wall of the large intestine 70. As the propulsion assembly 22 has the condition checking device 60, the endless track device 40 and the distal end ring 54a pushed on the large intestine 70 are deformed resiliently to enter the viewing area of the imaging unit 14. The doctor or operator views the display panel 21 to observe entry of the condition checking device 60 in the viewing area, and can check the condition of the propulsion assembly 22 pushed on the large intestine 70. In response to this, he or she stops the propulsion assembly 22 or returns the propulsion assembly 22 at a predetermined amount. Then the propulsion assembly 22 is advanced. Note that it is possible to stop the propulsion assembly 22 and then pull and return the elongated tube 3 at a predetermined amount. Note that the sleeve-shaped view segment of the condition checking device 60 is constituted by the return run 66 of the endless track device 40 and the distal end ring 54a.

If a lesion is discovered during the imaging, the doctor or operator may enter a medical instrument suitable for the treatment through the proximal instrument opening 15, to treat the lesion by protruding the instrument from the distal instrument opening 12.

To unload the propulsion assembly 22 from the tip device 3a, the clamping device 46 is rotated in a counterclockwise direction by use of a tool. The clamping device 46 moves axially upon rotation, and releases the C-ring 47 from pressure. The diameter of the C-ring 47 is increased by its resiliency to leave its inner surface from the tip device 3a. Thus, the propulsion assembly 22 becomes easily removable from the endoscope.

In the above embodiment, the propulsion assembly 22 has the distal end ring 54a and the endless track device 40 as a condition checking device. Other condition checking devices can be used in forms different from the above embodiment. A second preferred embodiment is described hereafter. Elements similar to those of the above embodiments are designated with identical reference numerals.

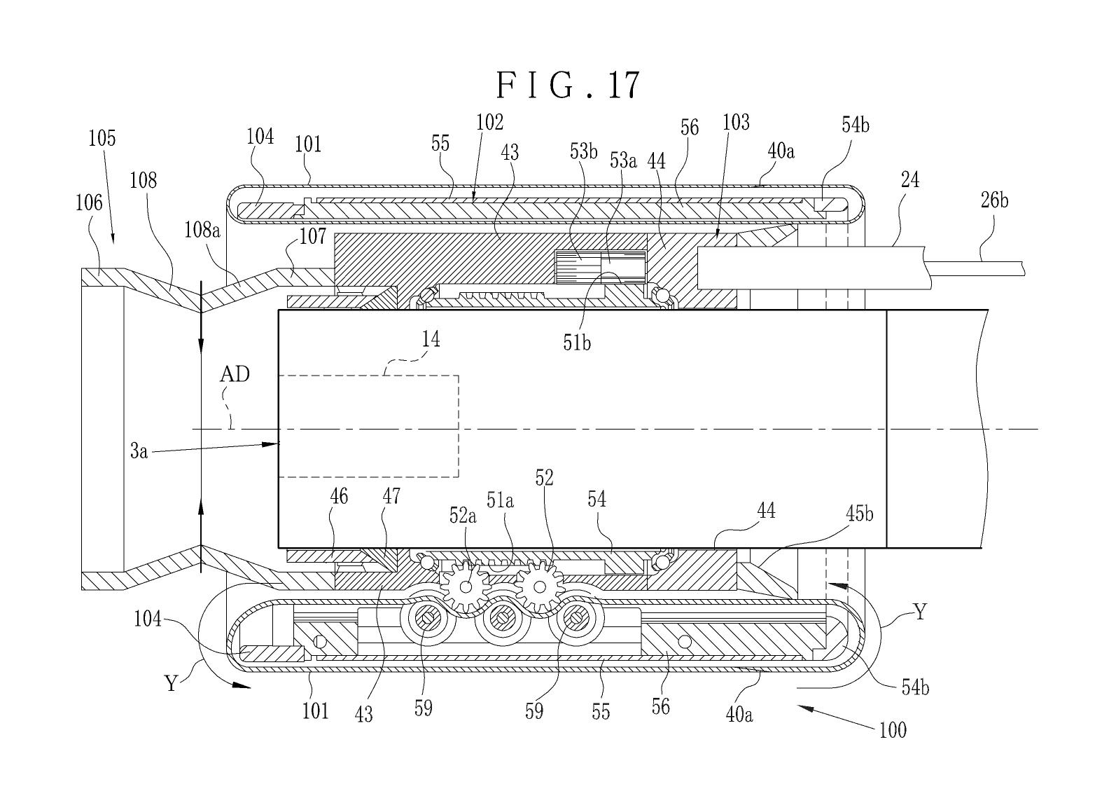

In FIG. 17, a propulsion assembly 100 for this purpose is illustrated, and includes an endless track device 101 (membrane), a barrel unit 102 or outer sleeve unit, and a drive unit 103 or inner sleeve unit. The barrel unit 102 supports the endless track device 101. The drive unit 103 is disposed between the endless track device 101 and the barrel unit 102. A distal end ring 104 is provided in the barrel unit 102 in place of the front end ring 54a of the above embodiment. The distal end ring 104 is cylindrical and attached to the distal end of the support sleeve 56. The endless track device 101 extends along inner and outer surfaces of the barrel unit 102 and endlessly moves in the axial direction in a manner similar to the endless track device 40.

A condition checking device 105 (end flange for visual aid) with a view segment (distal extension) is disposed with the drive unit 103 in place of the distal cover flange 45a of the above embodiment. The condition checking device 105 is disposed distally of the C-ring 47, and includes a distal end wall 106, a proximal end wall 107 and a neck portion 108. The end walls 106 and 107 have an equal outer diameter and an equal inner diameter. The neck portion 108 is disposed between the end walls 106 and 107. The condition checking device 105 is resilient, and formed from silicon rubber, fluororubber, polyurethane and the like. A distal end surface of the condition checking device 105 is disposed on a distal side from the endless track device 101. The neck portion 108 has a tapered wall 108a having a diameter decreasing at least from a proximal side toward a distal side. In the embodiment, the condition checking device 105 is positioned to align its axis with the axial direction of the tip device 3a upon mounting the propulsion assembly 100 on the tip device 3a. The axis of the neck portion 108 is aligned with the axis of the imaging unit 14. Note that the sleeve-shaped view segment of the condition checking device 105 is constituted by the distal end wall 106 and the neck portion 108.

The propulsion assembly 100 is mounted on the tip device 3a by positioning the condition checking device 105 on a distal side from the tip device 3a. When the condition checking device 105 is pushed on an inner wall of a body cavity, the neck portion 108 is deformed in the transverse direction resiliently to decrease the inner diameter, and enters a viewing area of the imaging unit 14 in a manner similar to the first embodiment. A doctor or operator can easily view the entry of the condition checking device 105 in the viewing area by observing the display panel 21.

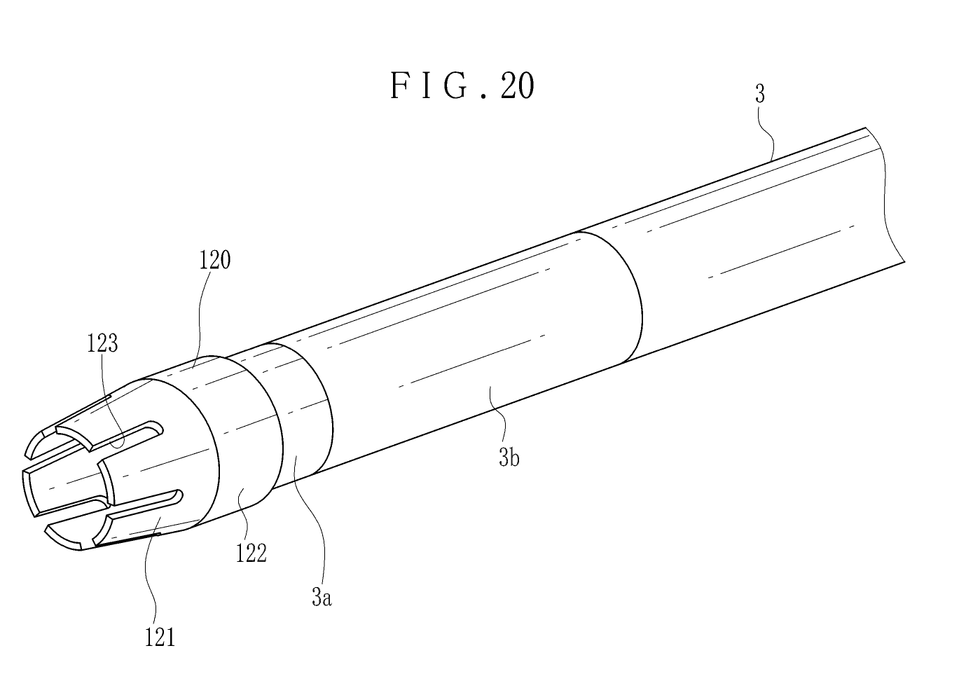

Also, it is possible in FIG. 17 to form slits in the condition checking device 105. See FIG. 20.

In the propulsion assembly 22 or 100 of the above embodiments, the condition checking device is deformable in the transverse direction. Another preferred embodiment is described now, in which an endless track device (membrane) constitutes a condition checking device.

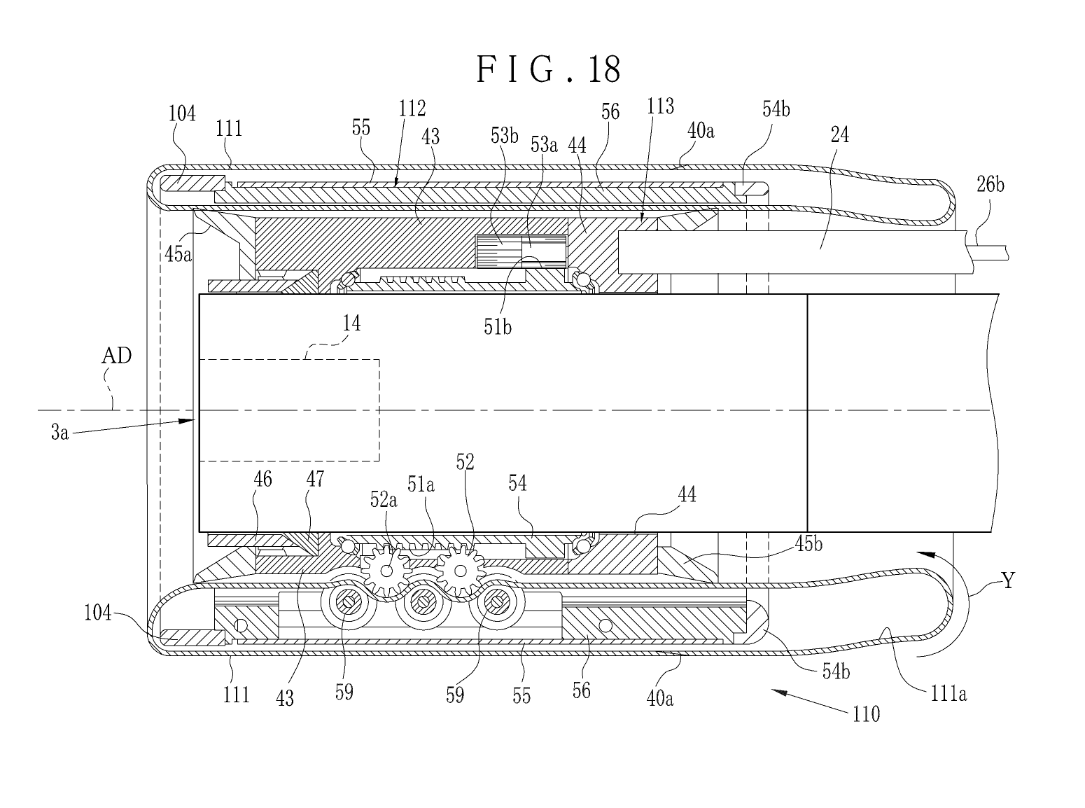

In FIG. 18, a propulsion assembly 110 of the third embodiment includes an endless track device 111 (membrane), a barrel unit 112 or outer sleeve unit, and a drive unit 113 or inner sleeve unit. The endless track device 111 is used also as a condition checking device. The barrel unit 112 supports the endless track device 111. The drive unit 113 is disposed inside the endless track device 111 and the barrel unit 112. The barrel unit 102 is repeated for the barrel unit 112. The drive unit 42 is repeated for the drive unit 113.

The endless track device 111 is in a bag shape to extend along the inner and outer surfaces of the barrel unit 112, and endlessly moves in the axial direction, in a manner similar to the endless track device 40 or 101 of the above embodiments. The endless track device 111 of the present example has an elongated form over the barrel unit 112 in the axial direction. The propulsion assembly 110 is fastened on the tip device 3a in a state of protruding the endless track device 111 on the distal side from the tip device 3a.

In FIG. 18, the endless track device 111 is not pushed on the inner wall of the body cavity. A loose portion 111a of the endless track device 111 is created on a proximal side of the barrel unit 112 at a size of a difference in the axial range from the barrel unit 112. The endless track device 111 is positioned on the proximal side with a sufficient inner space from the barrel unit 112. The endless track device 111 covers a distal end of the barrel unit 112 tightly. Therefore, the endless track device 111 is disposed outside the viewing area of the imaging unit 14 when the endless track device 111 is not pushed on the inner wall.

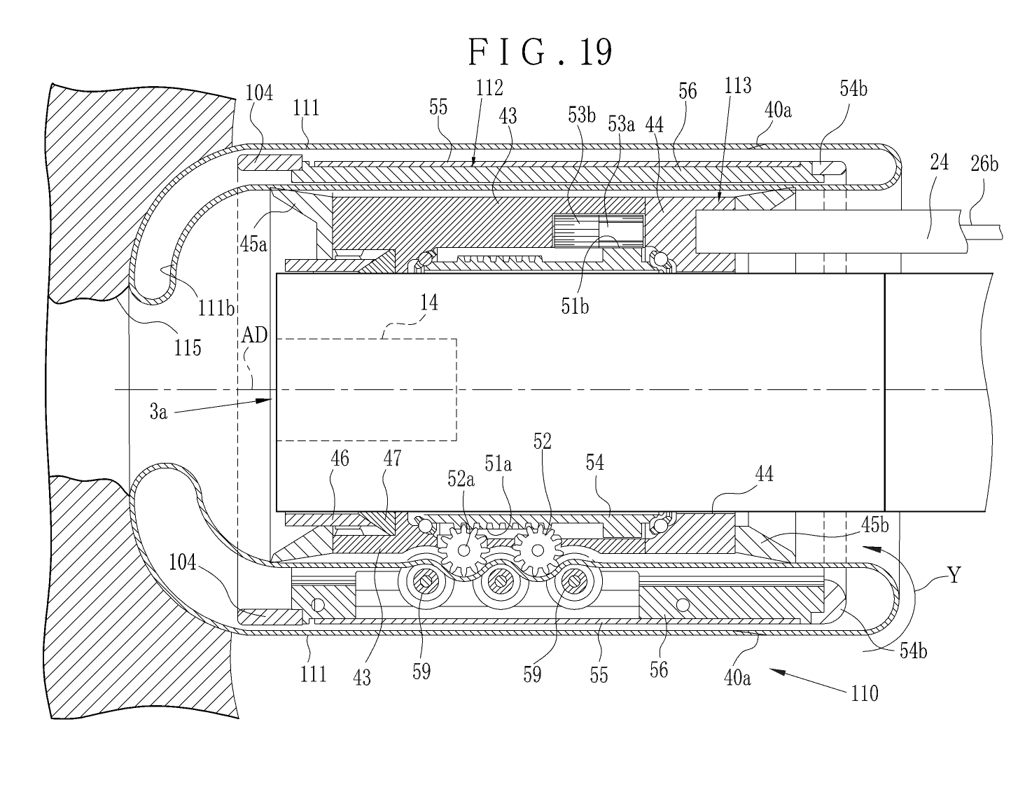

In FIG. 19, the endless track device 111 is pushed on an inner wall 115 of a body cavity. A distal portion of the endless track device 111 does not move quickly due to friction of the inner wall 115. The loose portion 111a at the proximal end moves in the distal direction. A loose portion 111b on an inner surface of the distal portion is created, and comes to enter the viewing area of the imaging unit 14. Consequently, it is possible to view the portion of the endless track device 111 in the viewing area on the display panel 21 as a sleeve-shaped view segment.

In the above embodiments, the condition checking device is included in the propulsion assembly for the tip device 3a. Another preferred condition checking device is a hood component for the tip device 3a of the endoscope as described below.

In FIG. 20, an endoscope hood component 120 for the elongated tube 3 is mounted on the tip device 3a for use. The hood component 120 includes a cylindrical support ring 122 and a tapered wall 121. The support ring 122 is fitted on the outside of the tip device 3a in a fixed manner.

The tapered wall 121 has a regular thickness, and is so tapered that its inner and outer diameters decrease gradually in the distal direction in contact with the support ring 122. Plural slits 123 are formed in the tapered wall 121 to extend in the axial direction on the distal side. The slits 123 are arranged at a pitch of a regular angle in a circumferential direction of the tapered wall 121. The tapered wall 121 is kept easily deformable by the slits 123 in directions transverse to the axial direction. The hood component 120 is fastened to the tip device 3a in a state of extending the tapered wall 121 on a distal side of the tip device 3a. Note that the sleeve-shaped view segment is constituted by the tapered wall 121.

Note that the tapered wall 121 can have a gradually decreasing thickness in a distal direction from a proximal side, and can be formed in a structure resiliently deformable from the inside in the transverse directions with a small rigidity in the bend. Furthermore, the tapered wall 121 can have a gradually decreasing thickness in a proximal direction from a distal side, and can be so formed that a shift of the distal portion is enlarged toward the inside in the transverse direction by enlarging the bend in the proximal portion. Note that distribution of the thickness of the tapered wall 121 is not limited to those examples, but can be determined suitably for various purposes.

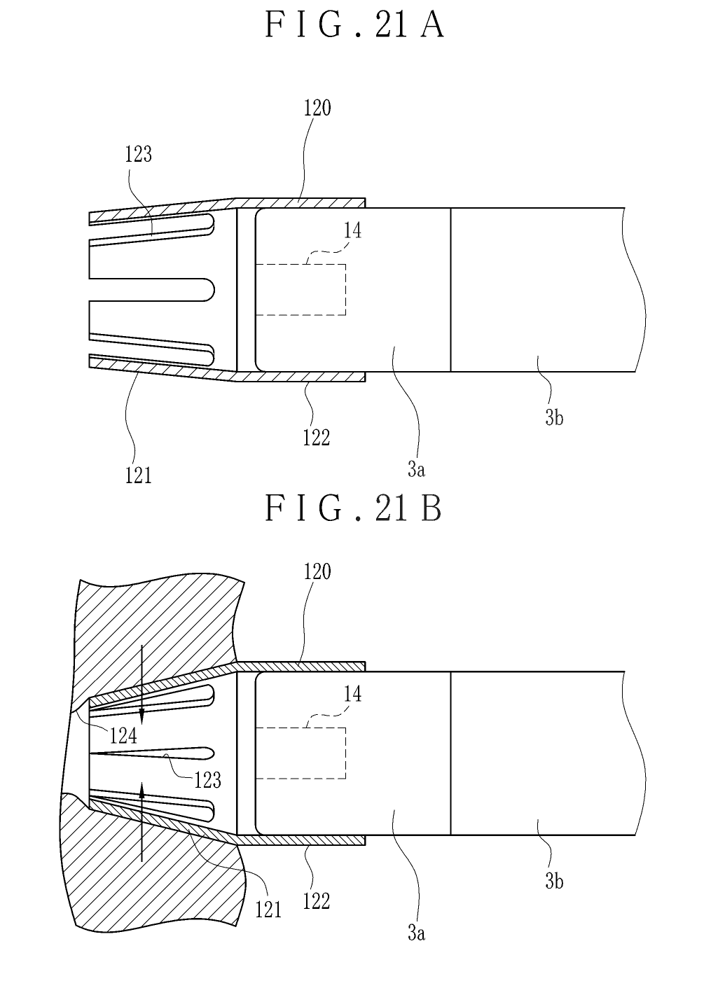

In FIG. 21A, the hood component 120 is not pushed on the inner wall of the body cavity. The tapered wall 121 is located outside a viewing area of the imaging unit 14. When the hood component 120 is pushed on an inner wall 124 of a body cavity, the tapered wall 121 is deformed radially to decrease its inner diameter upon decrease of the width of the slits 123. The tapered wall 121 enters the viewing area of the imaging unit 14. The doctor or operator views the display panel 21 to observe entry of the tapered wall 121 in the viewing area in a manner similar to the first to third embodiments.

Also, it is possible to form the hood component 120 in a neck shape. In other words, a tapered wall in the hood component 120 can be present only in a portion disposed on a proximal side of the tip device 3a.

In the above embodiments, the pushed condition is visually checked with the condition checking device upon entry of the elongated tube of the endoscope. However, the present invention is not limited to the above embodiments. It is possible to check the pushed condition at the time of the treatment of a lesion, as will be described with following variants of the embodiments.

This is specifically used for the time of invasive treatment to observe the pushed condition with the condition checking device, an example of the invasive treatment being the endoscope submucosal dissection (ESD) in which a mucosal lesion is found by imaging with the elongated tube 3 of the endoscope 2, and is dissected.

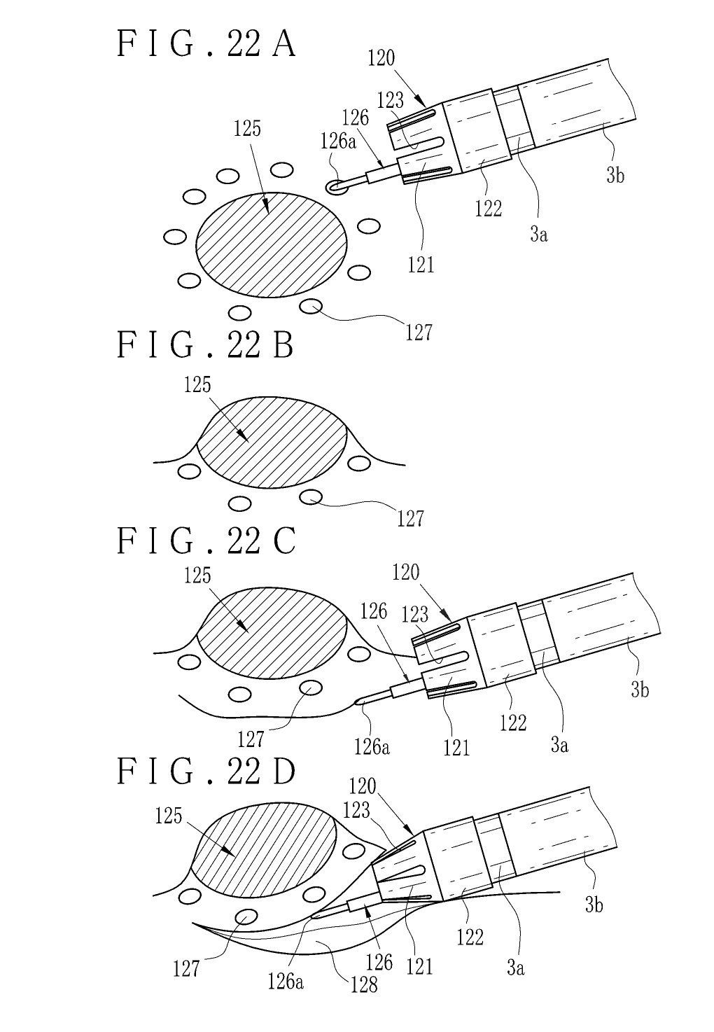

The hood component 120 is attached to the tip device 3a of the endoscope 2 for the purpose of the ESD procedure. In FIG. 22A, a doctor or operator creates indicia arranged around a mucosal lesion 125 which should be dissected. When the mucosal lesion 125 is discovered in the imaging, a high frequency surgical instrument 126 or high frequency scalpel is entered in the forceps channel in the endoscope 2 to protrude from the distal instrument opening 12. The display panel 21 is viewed, while an electrode 126a is set in contact with the surface of the mucosa, and supplied with a current of the high frequency. Portions of the mucosa on the electrode 126a are ablated, so that a plurality of indicia 127 or marking are formed on the mucosa. Then the high frequency surgical instrument 126 is pulled out of the forceps channel. A local injection apparatus (not shown) is punctured in the forceps channel instead of the high frequency surgical instrument 126. An injection needle is used to inject a fluid of a drug. As a result, the mucosal lesion 125 becomes swelled and protruded as illustrated in FIG. 22B. When the mucosal lesion 125 is enlarged sufficiently, the local injection apparatus is pulled out of the forceps channel. Then the high frequency surgical instrument 126 is penetrated again. In FIG. 22C, a current of high frequency is supplied to the electrode 126a of the high frequency surgical instrument 126. When the elongated tube 3 of the endoscope 2 is moved or the steering wheels 16 are rotated by manipulation, the electrode 126a of the high frequency surgical instrument 126 is moved along the indicia 127 to incise and peel the mucosal lesion.

It is possible in the course of the ESD to press the tip device 3a on a portion between a fascia 128 under the mucosal lesion 125 and the mucosal lesion 125. FIG. 22D illustrates this state for peeling the mucosal lesion 125 by advancing the elongated tube 3 of the endoscope 2. Bleeding may occur from the tissue after dissecting the mucosal lesion 125. The tip device 3a and the hood component 120 must be pushed slowly at a suitable pressure. When the hood component 120 is pushed on the inner wall as described above, the tapered wall 121 is resiliently deformed radially to decrease its inner diameter. He or she can view the display panel 21 to see the entry of the tapered wall 121 in the viewing area. When the hood component 120 enters the viewing area, he or she reduces force for thrusting the elongated tube 3, as the pushed condition of the tip device 3a and the hood component 120 can be monitored. Thus, the mucosal lesion 125 can be peeled by pushing the tip device 3a and the hood component 120 on the portion between the mucosal lesion 125 and the fascia 128 under the mucosal lesion 125 with suitable force.

Note that the inclination of the tapered wall of the above embodiments can be preferably determined so as to facilitate deformation of the condition checking device 60, 105 or 120, and facilitate local entry of the condition checking device 60, 105 or 120 in the viewing area 65 of the viewing window portion 10.

In the above embodiments, the endoscope is for a medical use. However, an endoscope of the invention can be one for industrial use, a probe of an endoscope, or the like for various purposes.

Although the present invention has been fully described by way of the preferred embodiments thereof with reference to the accompanying drawings, various changes and modifications will be apparent to those having skill in this field. Therefore, unless otherwise these changes and modifications depart from the scope of the present invention, they should be construed as included therein.

What is claimed is:

1. A condition checking device for an endoscope having a tip device for entry in a body cavity, and a viewing window portion formed in said tip device, comprising:
a sleeve-shaped view segment, disposed on a distal side in an axial direction, for resiliently deforming in a transverse direction crosswise to said axial direction when pushed on an inner wall of said body cavity, to enter a viewing area of said viewing window portion.

2. A condition checking device as defined in claim 1, comprising a propulsion assembly for constituting said view segment, and exerting force of propulsion to said tip device, for assistance to entry in said body cavity.

3. A condition checking device as defined in claim 2, wherein said propulsion assembly includes:
a coupling device for mounting on said tip device;
a support sleeve disposed around said coupling device;
a resiliently deformable endless track device for endlessly moving in said axial direction of said support sleeve by extending along inner and outer surfaces of said support sleeve.

4. A condition checking device as defined in claim 3, wherein said view segment includes a resilient end ring, disposed distally of said support sleeve, covered by said endless track device, and having a tapered wall of which a diameter decreases in said axial direction from said support sleeve.

5. A condition checking device as defined in claim 4, wherein said end ring is in a neck shape and includes a distal end wall, formed on a distal side of said tapered wall, and having a diameter increasing in said axial direction.

6. A condition checking device as defined in claim 3, wherein said view segment includes a resilient end ring, disposed distally of said coupling device, and having a tapered wall of which a diameter decreases in said axial direction from said coupling device.

7. A condition checking device as defined in claim 6, wherein said end ring is in a neck shape and includes a distal end wall, formed on a distal side of said tapered wall, and having a diameter increasing in said axial direction.

8. A condition checking device as defined in claim 3, wherein said view segment is constituted by said endless track device of a bag shape formed to extend in said axial direction longer than said support sleeve.

9. A condition checking device as defined in claim 3, further comprising:
a motor; and
a rotatable wire component, having a first end portion rotated by said motor, and a second end portion coupled to said propulsion assembly for driving said propulsion assembly.

10. A condition checking device as defined in claim 1, comprising a hood component, mounted on said tip device, and having a tapered wall of which a diameter decreases in said axial direction from a proximal side.

11. A condition checking device as defined in claim 10, further comprising a slit, formed in said hood component from a distal edge thereof, to extend in said axial direction.
